(19) United States
(12) Patent Application Publication
(10) Pub. No.: US 2010/0121141 A1
(43) Pub. Date: May 13, 2010

(54) ENDOSCOPIC CUTTING AND DEBRIDING DEVICE MOUNTED ON A FLEXIBLE AND MANEUVERABLE TUBE EMPLOYING A FLUID-DRIVEN TURBINE

(76) Inventor: Michael Rontal, Farmington Hills, MI (US)

Correspondence Address: GIFFORD, KRASS, SPRINKLE,ANDERSON & CITKOWSKI, P.C, PO BOX 7021, TROY, MI, 48007-7021, US

(21) Appl. No.: 12/616,990

(22) Filed: Nov. 12, 2009

Related U.S. Application Data
(60) Provisional application No. 61/113,929, filed on Nov. 12, 2008.

(51) Int. Cl. A61B 1/018; A61B 1/12; A61B 1/07

(52) U.S. Cl. 600/106

(57) ABSTRACT

An endoscope for visualizing and operating upon a surgical site within a human body employs an elongated tubular body adapted to be inserted through bodily passages so that the distal end is adjacent the surgical site. The body has a flexible section, at least proximal to the distal end, and manually adjustable bending controls at the proximal end may be manipulated to impose bending forces on the distal end to steer the distal end to the surgical site. A fluid turbine supported for rotation adjacent the distal end is powered by fluids pumped through a passage so as to rotate the turbine. The various cutters and debriding tools may be attached to the distal ends so that they are rotated by the turbine.

### CROSS-REFERENCE TO RELATED APPLICATION

This application claims priority of U.S. Provisional Patent Application Ser. No. 61/113,929 filed Nov. 12, 2008, which is incorporated herein by reference.

### FIELD OF THE INVENTION

This invention relates to endoscopes, laparoscopes or the like, and more particularly to a steerable, endoscopically guided fluid turbine powered device with detachable distal tip tools for cutting and burring that can be passed through body cavities to affect cutting and grind/milling of either hard or soft tissue.

### BACKGROUND OF THE INVENTION

In certain surgical procedures performed within passages of the human body, it may be necessary to remove bone or bard tissue by a grinding or milling process as well as removing softer tissue with a debriding instrument. For example, in sinus surgery it is often necessary to debride soft tissue overlying a bony structure and then grind away portions of the bone to remove infected areas. These surgical operations were originally performed using manual instruments which had to be inserted through bodily passages to the surgical site and then manipulated by the surgeon without the benefit of any image of the surgical site to remove the infected areas. More recently, elongated tubular endoscopes with steerable end sections have been employed for these operations. These endoscopes include optical systems which allow the physician to view the operating site from the proximal end of the instrument, outside of the body. Certain of these devices have employed illumination systems comprising light sources at the proximal end, usually LEDs, which pass light through optical fibers extending at least partially through the endoscope tube to illuminate the surgical site at the distal end, and imaging optics feeding return fibers which allow the surgeon to visualize the surgical site from the proximal end.

The use of optical fibers eliminates the need to transmit electrical currents through the endoscope to power the illumination of the surgical site by means of an electrically energized illumination source disposed at the distal end. The passage of electric currents through the endoscope always creates the possibility of accidental introduction of the electrical currents to the area of the surgical site.

However, when rotary powered surgical instruments such as mills, grinders, or debriders must be powered, either electrical currents must be introduced to the distal end through the endoscope to power electric motors or rather cumbersome rotary shafts must carry power from the proximal to the distal ends of the device. This may be relatively easy to do if the instrument is rigid, but flexible, steerable endoscopes can reach surgical sites unattainable by rigid instruments.

### SUMMARY OF THE INVENTION

The present invention overcomes these problems by providing an endoscope with a flexible section that may be manipulated from the proximal end by the surgeon to steer the instrument to a desired surgical site in which no electrical power is provided to the distal end.

In a preferred embodiment of the invention these goals are achieved by providing a fluid powered rotary turbine adjacent the distal end and powering the turbine by fluids pumped through a flexible lumen in the endoscope tube. The turbine shaft is connected to selected surgical cutters, mills, grinders and the like which operate on the surgical site.

In a preferred embodiment of the invention, which will be subsequently disclosed in detail, the turbine shaft powers the operating surgical instruments through a detachable connection which allows different surgical tools to be connected to the endoscope. For example, a debrider of the type employing an interior cutting tube mounted within an outer cutter housing may be used to remove soft tissue. The inner cutter member may be hollow and connected to a source of suction introduced from the proximal end, which passes through a second lumen in the endoscope. The fluid which turns the turbine may also be used to cool the surgical cutter or bathe the surgical area, with biofilm reducing agents or the like.

In a preferred embodiment of the invention, the control of the bendable distal end of the endoscope tube to allow the surgeon to steer the endoscope through bodily passages is achieved by employing two pairs of guide wires which extend through the length of the endoscope and may be manually manipulated by the surgeon to impose steering forces on the distal tip. One pair of diametrically opposed guide wires may be used to steer the end in a first direction and another pair, rotated by 90 degrees with respect to the first pair, may be used to manipulate the end in an orthogonal direction. Either individual control levers may be used for the two wire pairs or a single joystick-type arrangement may be used to manipulate all four guide wires simultaneously.

### BRIEF DESCRIPTION OF THE DRAWINGS

Other objects, advantages and applications of the present invention will be made apparent by the following detailed description of preferred embodiments of the invention. The description makes reference to the accompanying drawings in which:

FIG. 1 is a perspective view of a preferred embodiment of the surgical endoscope of the present invention being used to operate upon the sinus in patient's head illustrated in cross section;

FIG. 2 is a cross sectional view of the distal end of the endoscope of FIG. 1 with a debriding cutter attached to the end;

FIG. 3 is a cross sectional view similar to FIG. 2 in which the debriding attachment is separated from the distal end of the endoscope;

FIG. 4 is a cross-sectional view through the distal end of the endoscope of FIG. 1 with a burring or milling attachment joined to the distal end and connected to the shaft of a fluid turbine; and

FIG. 5 is a cross-sectional view through a distal end of an alternative embodiment of the endoscope with a debriding attachment of the type illustrated in FIG. 2 connected.

### DETAILED DESCRIPTION OF A PREFERRED EMBODIMENT

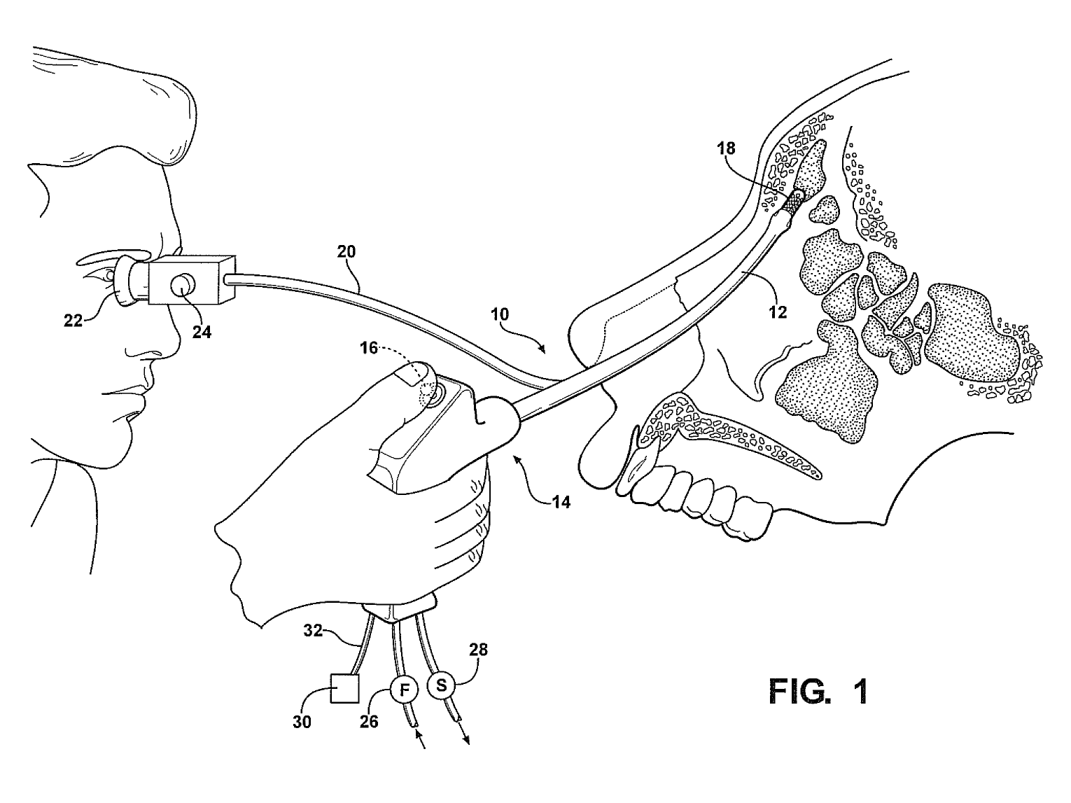

Referring to the drawings, and FIG. 1 in particular, a surgical endoscope forming a preferred embodiment of the invention, generally indicated at 10, comprises an elongated, tubular section 12 supported by a surgeon at the proximal end with a handle member, generally indicated at 14. The handle 14 is of the pistol grip type having a thumb actuated joystick 16 which the surgeon may use to bend the distal end of the tube 12 to guide it through passages to the operating site, in this case constituting a frontal sinus. The site is then operated on by a rotary tool 18 which is illustrated as a debriding tool in FIG. 1.

A fiber optic bundle 20 extends through the inside of the tube 12 from a point slightly separated from the proximal end. It ends at an ocular eyepiece 22 which may include appropriate controls such as a dioptric adjustment control 24. It could further include a photo electric converter which converts the optical signals passing through the fiber optic bundle 20 to an electrical signal which could be connected to a video display (not shown), or, alternatively to a radio transmitter for wirelessly connecting to a video display.

The proximal end 14 of the endoscope tube 12 connects to a fluid pump 26 and a suction pump 28 which are respectively connected to a source and a sump for a fluid (not shown), typically water. The fluid may include various additives such as biofilm reducing agents, antiseptics and the like. The pump 26 is adapted to the pump fluid from the source into a lumen in the endoscope tube 12 and the suction device 28 removes the fluid from the distal end of the endoscope tube through a second lumen.

A light source, preferable a LED 30, is connected to a fiber optic bundle 32 which extends through the handle into the endoscope tube 12.

The endoscope tube 12 is either flexible along its entire length or has a flexible section near the distal end, which may be controlled by the joystick 16 to form a gradual bend in the distal end to steer the endoscope through nonlinear bodily passages during insurtion to the surgical site as well subsequently described in more detail.

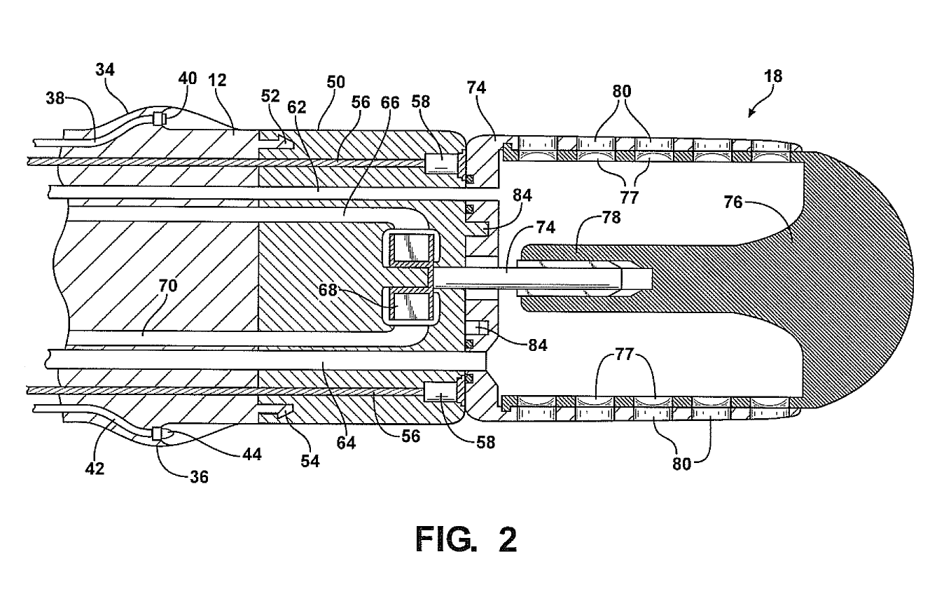

FIG. 2 illustrates a cross section through the endoscope tube adjacent to the distal end. A debriding cutter 18 detachably connected to the distal end of the tube 12. The tube 12 includes a pair of lobes 34 and 36 which extend outwardly from diametrically opposed points on the distal end of the tube and house the termination of two fiber optic bundles 38 an 40. The lobe 34 receives the distal termination of a fiber optic bundle 38, which is an extension of the fiber optic bundle 32 which is connected to the light source 30. It preferably has a lens 40 connected on its distal end which acts to illuminate the operating site for the surgeon's viewing. The lobe 36 carries the termination of a second fiber optic bundle 42 which forms an extension of the bundle 20 which connects to the eyepiece 22. It similarly terminates in a collecting lens 44. The lens transmits an image of the surgical site illuminated by a light from the lens 40 back to the eyepiece 22.

A turbine housing 50 is detachably coupled to the distal end of the tube 12 by connectors 52 and 54 so as to be removable for maintenance purposes.

Four cables 56, two of which are visible in the cross section of FIG. 2, extend through the endoscope tube 12 and the turbine housing 50, at 90 degree spacings to one another. Each of the cables 56 terminates in an attachment 58 at the distal end of the turbine housing 50. These cables 56 are used to bend the distal end of the endoscope tube 12 under control of the joystick 16. By manipulating the joystick 16 in a particular direction, the surgeon pulls on certain cables 56 and relaxes the tension on others, so as to impose a bending force on the tube. The joystick allows the tube end to be manipulated in a selected direction.

The particular manner of steering of the distal end of the endoscope is not critical to the present invention and other systems, such as any of the systems disclosed in patents classified in class 600, subclass 139 of the U.S. patent classification system, such as U.S. Pat. Nos. 7,311,659; 7,169,105; or 7,044,906 may be employed in other embodiments of the invention.

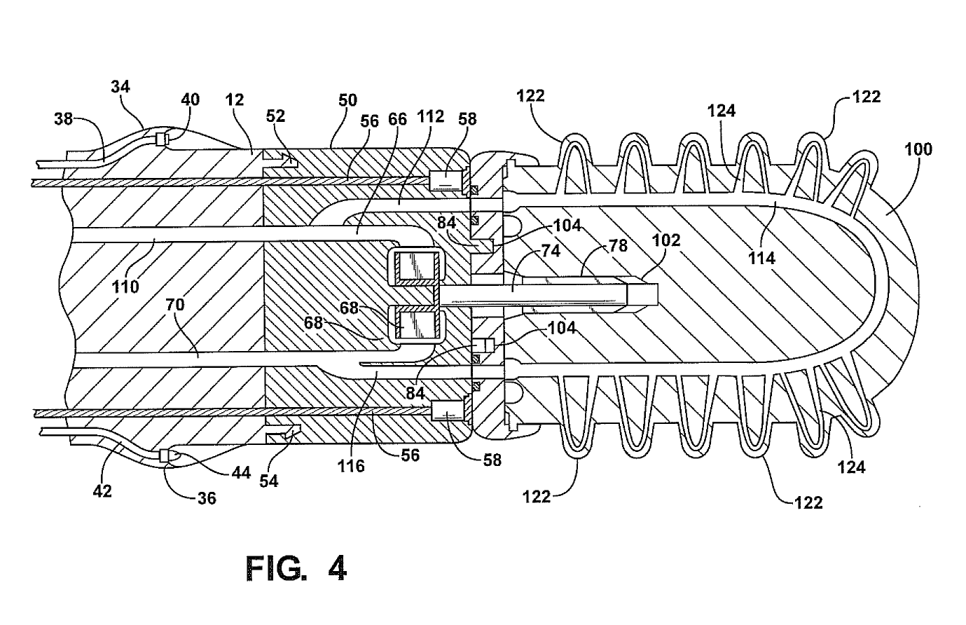

The endoscope has two lumens extending through its length for carrying fluid provided by the pump 26. One of the lumens, 62, extends through the turbine housing 50 to provide cooling fluid to the debriding tool 18 or whatever other tool may be connected, such as the burr illustrated in FIG. 4. Another lumen 64, evacuates the cooling fluid under forces imposed by the suction pump 28, and returns them to a sump. A third lumen 66 is connected to the pump 26 at the proximal end of the endoscope is 66 which provides pressured fluid to a rotary turbine 68 rotatably supported in the turbine housing 50. The fluid exiting the turbine 68 returns through a suction passage 70 under force of the suction pump 28.

The turbine 68 rotates a shaft 74 which extends beyond the distal end of the turbine housing 50 and drives rotary tools which may be attached to the distal end of the turbine head. In the case of 52 this constitutes a debrider 18. The debrider 18 has a stationary section 74 and a rotatable section 76. The rotatable section 76 has a coupling 78 with a female opening that slips over the shaft 74 of the turbine and is thus rotated by the turbine. The rotatable section 76 has a plurality of cutters 78 disposed about its periphery which pass over openings 80 spaced along the stationary section 74 of the debrider. Tissue is caught within the openings 80 and cut off by the cutters 78 in the same manner as an electric razor. The stationary debrider section 74 connects to the end of the turbine housing 50 by means of connectors 84 which extend outwardly from the turbine housing and lock into suitable connectors in the stationary section 74 of the debrider.

In use, the surgeon manually guides the endoscope tube 12 through body passages, using the joystick 16 to bend the end of the tube and bring the debrider 18 into contact with tissue that the surgeon desires to remove. The interior of the debrider is flushed with fluid coming through the passage 62 and the return fluid and debris from the debriding operation are extracted through the passage 64 to the suction pump 28. This system may incorporate an appropriate filter (not shown) to remove the debris from the returning fluid. The speed at which the rotary section of the debriders turn might be adjusted by controlling the flow from the pump 26 through manual controls associated with the hand piece 14.

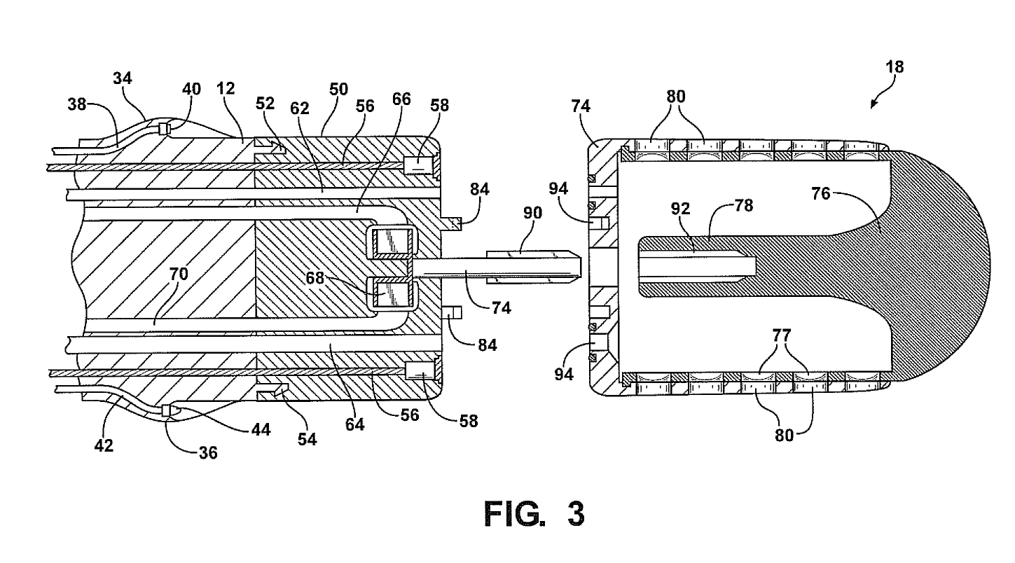

FIG. 3 illustrates the manner in which the debrider 18 may be attached to and removed from the endoscope tube 12 and the turbine drive. The turbine shaft 74 has radially extending flanges 90 which engage slots 92 formed in the female opening of the debrider rotary section 78. The extending sections 84 on the distal end of the turbine housing 50 lock within slots 94 formed in the stationary section 74 of the debrider 18 to secure the debrider to the turbine housing.

FIG. 4 illustrates an alternative embodiment of the invention supporting a burr or milling head 100 attached to the turbine housing 50 at the distal end of the surgical endoscope tube 12. The burr or milling cutter 100 has a female slot 102 which accepts the driving shaft 74 of the turbine 68. It also has receptors 104 adapted to receive the extending sections 84 at the distal end of the turbine housing 50. The endoscope tube has a flow line 110 that connects at the proximal end of the endoscope to the pump 26 and drives a turbine 68. Rather than providing a separate flow line like the flow line 62 shown in FIG. 2, the line 110 taps off into a flow line 112 which connects to a cooling passage 114 that passes through the burr 100 and returns through a tap off passage 116 to the return flow line 70 in the endoscope and the turbine housing.

The burr or milling head 100 comprises a number of cutter blades 122 extending about its outer periphery. These blades take any conventional form such as spiral or the like. The blades are water cooled by relatively small diameter passages 124 connecting the roots of the blades to the cooling fluid passage 114.

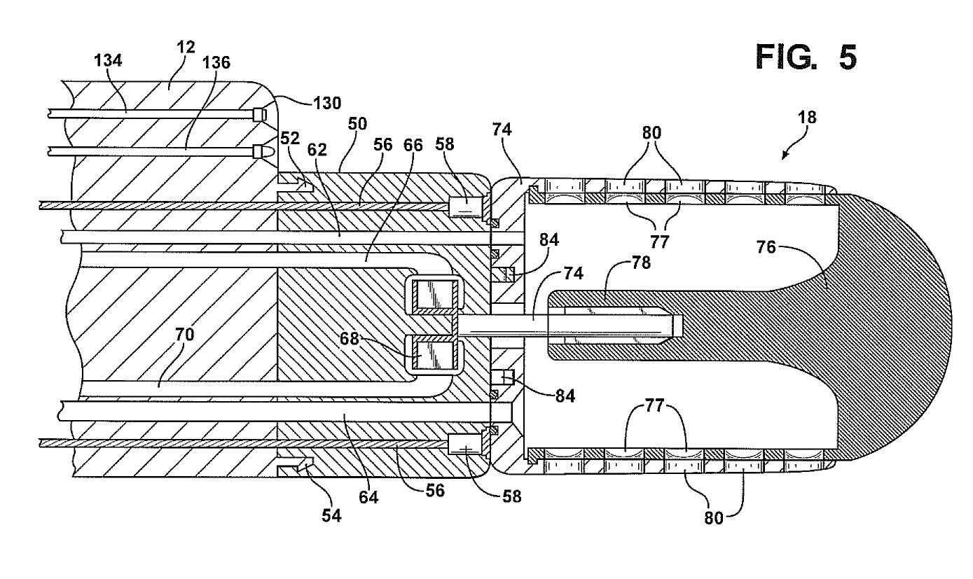

FIG. 5 illustrates an alternative embodiment of the surgical endoscope which only differs from the version of FIG. 2 in that the turbine housing 50 is smaller in diameter than the distal end 130 of the endoscope tube 12 and the turbine housing 50 is not centrally located but with respect to the surface 130 but is formed with one of its peripheral edges tangential to a peripheral edge of the tube section 130. This allows a section of the end 130 to avoid obstruction by the turbine housing 50 so that the two fiber optic bundles 134 and 136, which respectively convey the light from the LED 30 and captured an image of the surgical site for transmission back to the eyepiece 22 through the fiber optic bundle 20, are exposed to the surgical site at one side of the turbine housing 50. Otherwise FIG. 5 is identical to FIG. 2.

Having thus described my invention, I claim:

1. A surgical endoscope adapted to be inserted through a mammalian cavity into contact with interior organs to cut and/or remove tissue at a surgical site, comprising:
an elongated tubular body having a distal end and a proximal end, and having a flexible section at least proximal to the distal end;
manually adjustable bending controls supported at a distance from the distal end of the body operative to impose forces on the distal end, upon adjustment of the bending controls, to effect bending of the flexible section;
a fluid turbine supported for rotation at the distal end of the body;
first and second fluid passageways extending through the length of the body to the distal end;
a pump for introducing fluid under pressure into one of the fluid passageways so that fluid exiting that passageway at the distal end of the body turns the turbine and thereafter is removed from the distal end through the other fluid passageway;
a rotary surgical cutter supported at the distal end of the body and adapted to be rotated by the turbine to operate on a surgical site; and
optical fibers extending at least partially through the body to the distal end adapted to provide an image of the surgical site at a distance from the distal end;

2. The endoscopic surgical device of claim 1, further comprising suction means connected to the proximal end of the second fluid passage to draw fluid from the distal end through the passage.

3. The surgical endoscope of claim 1 wherein the optical fibers comprise a pair of optical fibers extend at least partially through the body to the distal end and a first of the pair transmits light from a source at the proximal end of the body to a lens at the distal end to illuminate the surgical site and a second of the pair collects light reflected from the surgical site to provide an image of the surgical site at a distance from the surgical site.

4. The surgical endoscope of claim 3 wherein said image of the surgical site is displayed on an optical eyepiece.

5. The surgical endoscope of claim 1 wherein the rotary surgical cutter comprises cutter blades and the endoscope includes fluid passageways for receiving fluid under pressure and carrying the fluid to the cutter blades.

6. The surgical endoscope of claim 1 wherein the support for the rotary surgical cutter comprises a separable fastener for joining the cutter to the distal end of the body, whereby, alternative surgical cutters may be supported on the distal end of the body.

7. The surgical endoscope of claim 1 wherein the manually adjustable bending controls for the distal end of the body comprise a plurality of cables each having one end joined to one of a plurality of spaced points at the distal end of the body.

8. The surgical endoscope of claim 6 wherein each of the cables has its end opposite to the end joined to a point on the distal end of the body joined to a manually actuable joystick supported at the proximal end of the body.

9. In an endoscope of type comprising an elongated tubular body having proximal and distal ends and a flexible section adapted to be inserted into a body cavity so its distal end is proximate a surgical site, comprising manually adjustable bending controls supported at the proximal end of the body operative to impose forces on the distal end to effect bending at the flexible section and optical fibers extending at least partially through the body to the distal end adapted to provide an image of the surgical site at a distance from the distal end, the improvement comprising:
a rotary surgical cutter supported on the distal end of the body;
a fluid turbine supported for rotation at the distal end of the body;
first and second fluid passageways extending through the body to the fluid turbine;
a pump for introducing fluid under pressure into one of the fluid passageways to the fluid turbine to rotate the turbine; and
a connection between the turbine and the surgical cutter so that rotation of the turbine rotates the surgical cutter.
